(19) United States
(12) Patent Application Publication
(10) Pub. No.: US 2019/0206568 A1
(43) Pub. Date: Jul. 4, 2019

(54) Motion Control Therapy Software for Evaluation and Development of Cognitive and Motor Functions

(71) Applicant: Kevin Clark, Westport, CT (US)

(72) Inventor: Kevin Clark, Westport, CT (US)

(21) Appl. No.: 15/859,562

(22) Filed: Dec. 31, 2017

(51) Int. Cl. G16H 50/20; G16H 20/30; G06N 99/00; G06K 9/00

(52) U.S. Cl. CPC G16H 50/20; G06K 9/00342; G06N 99/005; G16H 20/30

(57) ABSTRACT

A system for diagnosing, evaluating, and treating cognitive and motor function disorders, injuries, and diseases by capturing and analyzing body movements in reaction to various stimuli and requests in interactive virtual environments through a motion detection camera and motion control software. The system analyses patterns in cognitive and motor body movement to predict certain diagnostic or therapeutic outcomes as well as to highlight the efficacy of certain techniques in regards to a patient's treatment.

### FIELD OF THE INVENTION

The field of the invention relates to a system for diagnosing, evaluating, and treating cognitive and motor function disorders, injuries, and diseases by capturing and analyzing body movements in reaction to various stimuli and requests in interactive virtual environments through a motion detection camera and motion control software.

### BACKGROUND OF INVENTION

Cognitive and motor function disorders, injuries, or diseases can cause learning, problems, memory loss, difficulties with problem solving, uncontrollable movements, and difficulties with everyday physical movements.

Diagnosing and treating cognitive and motor function disorders or diseases symptoms, requires extensive evaluation, development of individualized treatment plans, periodic re-evaluation of treatment effectiveness, and adjustments to the treatment plans.

These processes require extensive, and accurate record keeping of the patient's symptoms before and after treatments, which is time-consuming, expensive, and can be quite imprecise between different treatment providers.

Furthermore, patients in rural areas, those with busy schedules, and those with very limited physical mobility, find it more difficult to obtain extensive and periodic evaluation and treatment in a therapeutic center.

A system is needed that more accurately, and precisely, measures and evaluates a patient's symptoms due to a cognitive and motion function disorders or diseases, and evaluates specific treatments on those symptoms. A system that maintains highly accurate diagnosis and treatment results records to allow different therapy providers to assess, and re-assess treatment plans for more consistent results.

The system should be useable for in-home monitoring to allow patients to provide more extensive symptom data, and to allow therapy professionals access to patient symptom monitoring remotely.

### SUMMARY OF THE INVENTION

There are additional features of the invention that will be described hereinafter and which will form the subject matter of the claims appended hereto. In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of the description and should not be regarded as limiting

The subject invention discloses a diagnosis and evaluation system for body motor function disorders, the system comprising: a computing device comprising executable software; a data storage device; a first database of body movements on the data storage device, wherein the first database of body movements corresponds to body movement symptoms of at least one body motor function disorder; a motion detection camera, wherein the motion detection camera detects a user, wherein the user is subjected to a plurality of stimuli in an interactive virtual environment to generate a plurality of body movements from the user, further wherein the motion detection camera captures the plurality of body movements from the user for storage onto a second database of body movements on the data storage device; wherein the system compares the second database of body movements from the user's plurality of body movements to the first database of body movements to determine if the user is diagnosed with a body motor function disorder, and further wherein the system outputs a unique evaluation of the user's body function performance.

In embodiments of the subject invention, the motion detection camera captures a second plurality of body movements from the user's movements after at least one treatment session for storage on a third database of body movements on the data storage device, wherein the system compares the third database of body movements to the first and second database of body movements to determine improvement of the user's body motor function disorder symptoms, and further wherein the system outputs a second unique evaluation of the user's body function performance.

In embodiments of the subject invention, the motion detection camera captures a third plurality of body movements from the user's movements after at least two treatment sessions for storage on a fourth database of body movements on the data storage device, wherein the system compares the fourth database of body movements to the first, second, and third databases of body movements to determine improvement of the user's body motor function disorder symptoms, and further wherein the system outputs a third unique evaluation of the user's body function performance.

In embodiments of the subject invention, the motion detection camera captures an N plurality of body movements from the user's movements after at least N−1 treatment sessions, where N is an integer greater than 2, for storage on an N+1 database of body movements on the data storage device, wherein the system compares the N+1 database of body movements to the N database of body movements to determine improvement of the user's body motor function disorder symptoms, and further wherein the system outputs an N unique evaluation of the user's body function performance.

The subject invention discloses a diagnosis and evaluation system for body motor function disorders, the system comprising: a computing device comprising executable software; a data storage device; a first database body movements on the data storage device, wherein the first database of body movements corresponds to body movement symptoms of at least one body motor function disorder; a motion detection camera, wherein the motion detection camera detects a user, wherein the user is subjected to a plurality of stimuli in an interactive virtual environment to generate a plurality of body movements from the user, further wherein the motion detection camera captures the plurality of body movements from the user for storage onto a second database of body movements on the data storage device; wherein the system compares the second database of body movements from the user's plurality of body movements to the first database of body movements to determine if the user is diagnosed with a body motor function disorder, and further wherein the system outputs a first unique evaluation of the user's body function performance; wherein after at least N−1 treatment sessions, where N is an integer greater than 2, the motion detection camera captures an N plurality of body movements from the user's body movements in response to the plurality of stimuli in the interactive virtual environment for storage on an N+1 database of body movements on the data storage device, wherein the system compares the N+1 database of body movements to the N database of body movements to determine improvement of the user's body motor function disorder symptoms, and further wherein the system outputs an N unique evaluation of the user's body function performance.

The subject invention also discloses a diagnosis and evaluation system for body motor function disorders, the system comprising: a computing device comprising executable software; a data storage device; a first database of first plurality of body movements on the data storage device, wherein the first plurality of body movements corresponds to body movement symptoms of at least one body motor function disorder; a motion detection camera, wherein the motion detection camera detects a user, further wherein the motion detection camera captures a second plurality of body movements from the user's movements for storage on a second database of body movements on the data storage device; wherein the system compares the second database of body movements from the user's body movements to the first database of body movements to determine if the user is diagnosed with a body motor function disorder, and further wherein the system outputs output a unique evaluation of the user's body function performance.

The subject invention discloses a diagnosis and evaluation system for body cognitive function disorders, the system comprising: a computing device comprising executable software; a data storage device; a first database of first plurality of body movements on the data storage device, wherein the first plurality of body movements corresponds to body movement symptoms of at least one body cognitive function disorder; a motion detection camera, wherein the motion detection camera detects a user, further wherein the motion detection camera captures a second plurality of body movements from the user's movements for storage on a second database of body movements on the data storage device; wherein the system compares the second database of body movements from the user's body movements to the first database of body movements to determine if the user is diagnosed with a body motor function disorder, and further wherein the system outputs output a unique evaluation of the user's body function performance.

The subject invention discloses a diagnosis and evaluation system for body cognitive and motor function disorders, the system comprising: a computing device comprising executable software; a data storage device; a first database of first plurality of body movements on the data storage device, wherein the first plurality of body movements corresponds to body movement symptoms of a plurality of body cognitive and motor function disorders; a motion detection camera, wherein the motion detection camera detects a user, further wherein the motion detection camera captures a second plurality of body movements from the user's movements for storage on a second database of body movements on the data storage device; wherein the system compares the second database of body movements from the user's body movements to the first database of body movements to determine if the user is diagnosed with a body motor function disorder, and further wherein the system outputs output a unique evaluation of the user's body function performance.

In embodiments of the subject invention, the motion detection camera captures a third plurality of body movements from the user's movements after at least one treatment session for storage on a third database of body movements on the data storage device, wherein the system compares the third database of body movements to the first and second database of body movements to determine improvement of the user's body cognitive or motor function disorder symptoms, and further wherein the system outputs a second unique evaluation of the user's body function performance.

In embodiments of the subject invention, the motion detection camera captures a fourth plurality of body movements from the user's movements after at least two treatment sessions for storage on a fourth database of body movements on the data storage device, wherein the system compares the fourth database of body movements to the first, second, and third databases of body movements to determine improvement of the user's body cognitive or motor function disorder symptoms, and further wherein the system outputs a third unique evaluation of the user's body function performance.

In embodiments of the subject invention, the motion detection camera captures an N plurality of body movements from the user's movements after at least N−2 treatment sessions, where N is an integer greater than 2, for storage on an N database of body movements on the data storage device, wherein the system compares the N database of body movements to the N−1 databases of body movements to determine improvement of the user's body cognitive motor function disorder symptoms, and further wherein the system outputs a N−1 unique evaluation of the user's body function performance.

In embodiments of the subject invention, the computing device may be a game console, a laptop, a tablet, a smartphone, or a desktop computer.

In embodiments of the subject invention, the system can be used to diagnose, evaluate, and development treatment plans for cognitive disorders, injuries, and diseases, including but not limited to: cognitive disorders that affect learning, short-term memory loss, long-term memory loss, perception, or problem solving.

In embodiments of the subject invention, the system can be used to diagnose, evaluate, and development treatment plans for motor disorders, injuries, and diseases, including but not limited to: motor disorders that cause uncontrollable and involuntary body movements, such as tremors, jerks, twitches, spasms, contractions, or gait problems.

In embodiments of the subject invention, the system can be used to diagnose, evaluate, and development treatment plans for Autism Spectrum Disorder or Parkinson's Disease, or other motor diseases that affect muscle movement, control, coordination, or balance.

In embodiments of the subject invention, the system can be used to diagnose, evaluate, and development treatment plans for Ataxia, or other motor diseases that cause tremors, lack of coordination, loss of balance, instability, inaccuracy, clumsiness, gait problems, or speech problems.

In embodiments of the subject invention, the system can be used to diagnose, evaluate, and development treatment plans for Dystonia, Focal Dystonia, Multifocal Dystonia, Segmental Dystonia, or Hemidystonia.

In embodiments of the subject invention, the system can be used to diagnose, evaluate, and development treatment plans for Developmental coordination disorder, dyspraxia, or other motor diseases that affect planning of movements and co-ordination.

In embodiments of the subject invention, the system can be used to diagnose and evaluate cognitive and motor disorders, these assessment tools can include, but aren't limited to: International Cooperative Ataxia Rating Scale (ICARS), The Berg Balance Scale, Tandem Walking test, arm or leg tapping tests, and finger-body part testing.

In embodiments of the subject invention, the system can be used to diagnose and evaluate developmental coordination disorder, these assessment tools can include, but aren't limited to: Movement Assessment Battery for Children, Peabody Developmental Motor Scales (PDMS-2), Bruininks-Oseretsky Test of Motor Proficiency (BOTMP-BOT-2), Test of Gross Motor Development (TGMD-2, Maastrichtse Motoriek Test (MMT), Wechsler Adult Intelligence Scale (WAIS-IV), Wechsler Individual Achievement Test (WAIT-II), and Test of Word Reading Efficiency (TOWRE-2).

In embodiments of the subject invention, the system can be used to develop, evaluate, and re-fine treatment plans for or cognitive and motor disorders, injuries, and diseases, including but not limited to: using visual, auditory, and somatosensory cuing devices in conjunction with walking aids or other aids to improve physical movements.

In embodiments of the subject invention, the system can be used to develop, evaluate, and re-fine treatment plans for or cognitive and motor disorders, injuries, and diseases, including but not limited to: using physical therapy such as gait training, mobility training, strength training, static balance training, dynamic balance training, trunk-limb coordination, stairs training, contracture prevention, vestibular habituation, Frenkel exercises, proprioceptive neuromuscular facilitation (PNF), decomposition or slowing of multijoint movement therapy, constraint-induced movement therapy, therapeutic exercise, manual stretching, soft tissue and joint mobilization, postural training and bracing, neuromuscular electrical stimulation, activity and environmental modification.

In embodiments of the subject invention, the system can be used to develop, evaluate, and re-fine treatment plans for or cognitive and motor disorders, injuries, and diseases, including but not limited to: using occupational therapy such as training fine motor coordination, providing movement inhibitory techniques, teaching alternative methods of everyday activities such bathing, dressing, or toileting; Providing upper extremity splinting; repeating simple tasks to commit procedures to memory.

In embodiments of the subject invention, the term “substantially” is defined as at least close to (and can include) a given value or state, as understood by a person of ordinary skill in the art. In one embodiment, the term “substantially” refers to ranges within 10%, preferably within 5%, more preferably within 1%, and most preferably within 0.1% of the given value or state being specified.

In embodiments of the subject invention, the term “relatively” is defined as a comparison of a property, or the proportion of a property between two components.

There has thus been outlined, rather broadly, the more important features of the invention in order that the detailed description thereof that follows may be better understood, and in order that the present contribution to the art may be better appreciated. There are additional features of the invention that will be described hereinafter and which will form the subject matter of the claims appended hereto. These together with other objects of the invention, along with the various features of novelty, which characterize the invention, are pointed out with particularity in the claims annexed to and forming a part of this disclosure.

### BRIEF DESCRIPTION OF THE DRAWINGS

Advantages of the present invention will be apparent from the following detailed description of exemplary embodiments thereof, which description should be considered in conjunction with the accompanying drawings:

FIG. 1 is a top view of one embodiment of the system capturing a series of body movements of a patient for therapeutic evaluation or treatment.

FIG. 2 is a front view of the embodiment of the system capturing a series of body movements of a patient for therapeutic evaluation or treatment.

FIG. 3 is a block diagram illustrating a configuration of the system.

FIG. 4 is an embodiment of a screenshot of an evaluation of a patient.

### DETAILED DESCRIPTION OF EMBODIMENTS

While several variations of the present invention have been illustrated by way of example in particular embodiments, it is apparent that further embodiments could be developed within the spirit and scope of the present invention, or the inventive concept thereof. However, it is to be expressly understood that such modifications and adaptations are within the spirit and scope of the present invention, and are inclusive, but not limited to the following appended claims as set forth.

For a conceptual understanding of the invention and its operational advantages, refer to the accompanying drawings and descriptive matter in which there are preferred embodiments of the invention illustrated. Other features and advantages of the present invention will become apparent from the following description of the preferred embodiment(s), taken in conjunction with the accompanying drawings, which by way of example; illustrate the principles of the invention.

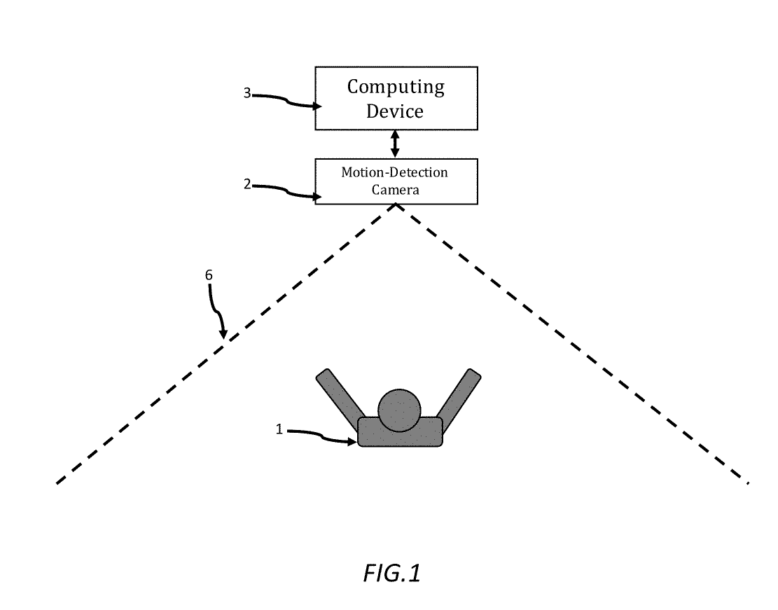

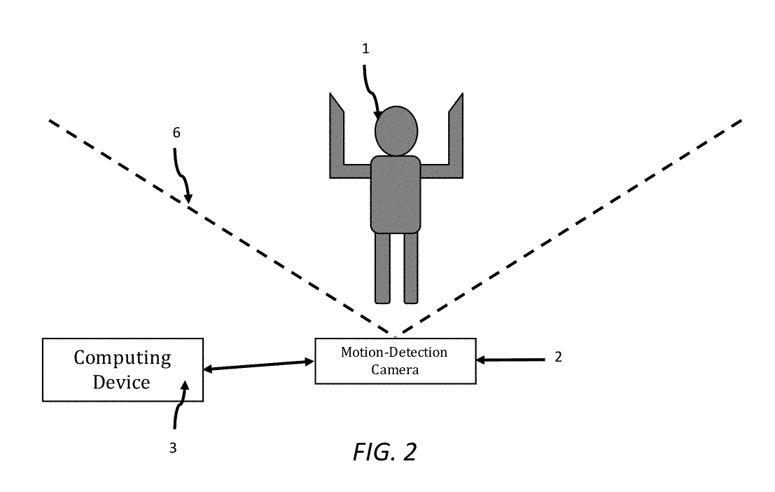

As illustrated in FIGS. 1-4, the subject invention discloses a system for diagnosing, evaluating, and treating cognitive and motor function disorders, injuries, and diseases by capturing and analyzing body movements through a motion detection camera and motion control software.

The system incorporates at least one motion-detection camera platform 2, and at least one computing device 3 capable of running executable software applications, such as a laptop, a tablet, a smartphone, or a desktop computer. The computing device 3 executes software that operatively connects the motion detection camera 2 with the computing device 3 and turns recognized body movements points from the motion detection camera into data points. In further embodiments of the subject invention, the system may include multiple motion-detection cameras.

The system consists of three stages: A) collection of patient 1 body movement data; B) storage of data; and C) analysis of data.

To begin, a medical professional, or health care provider creates or downloads a digital database of body movements that correspond to symptoms of cognitive and motor disorders onto a storage device 4 of the computing device 3. In embodiments of the subject invention, this database of body movements may be pre-installed onto the storage device 4 or computer-readable media that may be uploaded to the storage device 4. In another embodiment of the subject invention, the database of body movements may be downloaded from the Internet 5.

In embodiments of the subject invention, the medical professional may designate one or more designated body movements to begin and end the evaluation.

The height, weight, sex, and body type of a patient 1 are then inputted into the computing device 3.

The first step is for the patient 1 to step in from of the motion-detection camera platform 2, and allow the camera 1 to recognize a patient's body points. In embodiments of the subject invention, the system may recognize the constant X and Y axis of at least twelve body points on the patient 1: two feet, two knees, one Torso, two elbows, two shoulders, two Hands, and the head. In additional embodiments of the subject invention, the system may recognize the constant X, Y, and Z axis of at least twelve body points on the patient 1: two feet, two knees, one Torso, two elbows, two shoulders, two Hands, and the head.

The motion-detection camera platform 2 begins tracking and recording body movements of the patient 1. The motion detection camera 2 will detect 6 the patient's 1 body movements and, through the software, capture each movement into a second database of body movements. The motion detection camera 2 may identify different movements for each part of the body, including the head, each arm, and each leg.

In the Collection stage, the patient 1, is presented with a plurality of therapeutically related scenarios in which they need to interact in a specified way in front of the motion detection camera 2. These scenarios may consist of either: a) Cognitive cues, in which the patient 1 needs to decide how or when to react to a certain stimulus; or ii) Motor cues, in which the patient 1 needs to move in a specified way.

In embodiments of the subject invention, these cognitive or motor cues 7 may be defined by a therapist in conjunction with their regular therapeutic treatment.

The system collects movement of the patient 1 through the motion control camera 2 in relation to these cognitive or motor cues 7 in substantial real time. The body movements collected in the second database of body movements may include:

i. Patient 1 body movements demonstrating his or her reaction time to a specific stimulus;

ii. Patient 1 body movements demonstrating his or her decisions or choices made in a time-constrained environment;

iii. Patient 1 body movements demonstrating his or her ability to act or move as prompted by the program or by a therapist;

iv. Patient 1 body movements demonstrating his or her subconscious biases or changes in actions or behavior with repeated stimulus in varying environments;

v. Patient 1 body movements demonstrating the effect of different musical cues or characteristics, such as musical scale, timbre, or tempo, on his or her performance of various tasks;

vi. Patient 1 body movements demonstrating his or her level of engagement with various cues or tasks, especially in regards to the visual cues, musical cues, or therapeutic tasks;

vii. Patient 1 body movements demonstrating his or her physical range of motion for a given body part;

viii. Patient 1 overall physical activity levels;

ix. Patient 1 body movements demonstrating the distance traveled overall or for a given body part, along with the speed of the body movements;

x. Patient 1 body movements demonstrating his or her ability to perform certain physical gestures or movements, often in conjunction with a therapist's regimen; and

xi. Patient 1 body movements demonstrating his or her patterns among any of the above information or other data collected over periods of time.

These body movements are stored in the second database of body movements on the storage device 4.

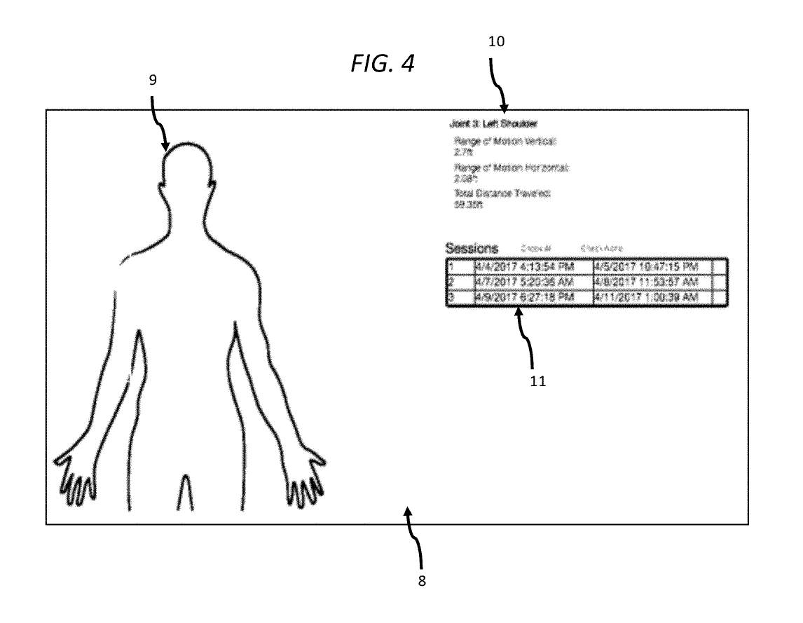

These recorded body movements may be provided to any therapist through an online interface. FIG. 4 illustrates an embodiment of the graphical user interface patient summary profile results screen 8. This GUI screen 8 displays recognized body points 9 of the patient 1, a summary of range motions for a selected body part 10, and a summary 11 of patient 1 sessions.

In analysis of data stage, the system applies a number algorithms 12 to analyze the patient's body movement, including, but not limited to: a) Comparing success and failure rates of the patient 1 body movements in response to cognitive cues, especially in relation to an environmental stimulus; b) Changes in the patient 1 body movements with a change in the environmental stimulus; c) Statistics about the patient 1 body movements during a specific session; d) General analysis of the information collected, especially in relation to other information within a given session inside the application; and e) comparing the patient's body movements to the first database of body movements that correspond to symptoms of cognitive and motor disorders

In embodiments of the subject invention, the system's learning algorithms include, but are not limited to: Decision Trees; Bayes Classification; Support Vector Machines; Hidden Markov Models; Ensemble methods; Unsupervised learning; Pattern discerning via wavelet analysis or other signal processing; or other algorithms related to classifying a patient performance and highlighting progress or relevant patterns.

The system then connects patterns in the data collected with positive or negative outcomes including, but not limited to: Improvement of a patient specific physical or cognitive condition over time; performing or confirming a medical diagnosis for the patient; monitoring changes in physical or mental performance for the patient; and monitoring changes in the patient memory or ability to learn, especially in regards to musical stimulus.

The system connects patterns in the patient data to predict the probability of specific outcomes for the patient. These patterns and probability are updated each time the patient inputs new body movement data.

Therapists can use the system as a diagnostic tool within their regular practice. Therapists can use this data to determine their patient's individual progress as well as to evaluate the efficacy of certain treatment techniques.

The invention provides patients and medical providers with a means of providing and gathering body movement data without requiring a pediatrician or health worker to be physically present to monitor progress. This invention allows medical providers to monitor and support health treatment, monitor quality of care of individuals with special needs in the community, and provide access to health supervision to individuals in rural areas, individuals who lack resources, and individuals that lack means of visiting a specialist.

Architecture of the System

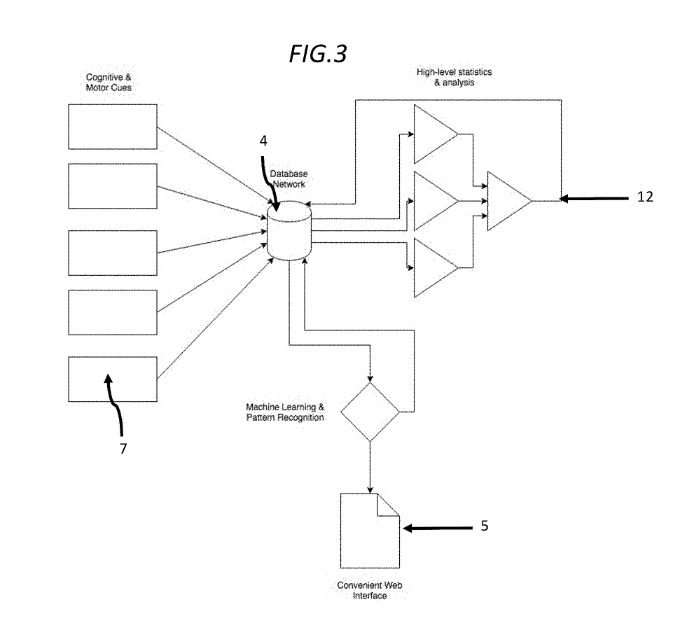

FIG. 3 illustrates a block diagram that depicts one embodiment of the computing device 3 architecture. The computing device 3 may include a communication device (such as a bus), a CPU/processor, a main memory, and a storage device 4.

The communication device may permit communication between the computing device 3 and the motion detection camera 2, and the Internet 5. Embodiments of the communication device of the computing device 3 may include any transceiver-like mechanism that enables the computing device 3 to communicate with other devices or systems. The communication may be over a network such as a wired or wireless network. The network communication may be based on protocols such as Ethernet, IP, TCP, UDP, or IEEE 802.11.

Embodiments of the processor unit of the computing device 3 may include processors, microprocessors, multi-core processors, microcontrollers, system-on-chips, field programmable gate arrays (FPGA), application specific integrated circuits (ASIC), application specific instruction-set processors (ASIP), or graphics processing units (GPU). In one embodiment, the processor unit may enable processing logic to interpret and execute instructions.

In a further embodiment, the main memory may store computer retrievable information and software executable instructions. These software executable instructions may be instructions for use by the processor unit. The storage device 4 may computer retrievable information and software executable instructions for use by the processor and may also include a solid state, magnetic, or optical recording medium.

Embodiments of an input terminal of the computing device 3 may include a keyboard, a mouse, a pen, a microphone combined with voice recognition software, a camera, a smartphone, a tablet, a touchpad, or a multi-point touch screen.

In embodiments of the subject invention, the underlying architecture of the system may be implemented using one or more computer programs, each of which may execute under the control of an operating system, such as Windows, OS2, DOS, AIX, UNIX, MAC OS, iOS, ChromeOS, Android, and Windows Phone or CE.

The many aspects and benefits of the invention are apparent from the detailed description, and thus, it is intended for the following claims to cover such aspects and benefits of the invention, which fall within the scope, and spirit of the invention. In addition, because numerous modifications and variations will be obvious and readily occur to those skilled in the art, the claims should not be construed to limit the invention to the exact construction and operation illustrated and described herein. Accordingly, all suitable modifications and equivalents should be understood to fall within the scope of the invention as claimed herein.

What is claimed is:

1. A diagnosis and evaluation system for body motor function disorders, the system comprising:
a computing device comprising executable software;
a data storage device;
a first database of body movements on the data storage device, wherein the first database of body movements corresponds to body movement symptoms of at least one body motor function disorder;
a motion detection camera, wherein the motion detection camera detects a user, wherein the user is subjected to a plurality of stimuli in an interactive virtual environment to generate a plurality of body movements from the user, further wherein the motion detection camera captures the plurality of body movements from the user for storage onto a second database of body movements on the data storage device;
wherein the system compares the second database of body movements from the user's plurality of body movements to the first database of body movements to determine if the user is diagnosed with a body motor function disorder, and further wherein the system outputs a unique evaluation of the user's body function performance.

2. The system of claim 1, wherein the system generates a unique treatment plan based on the user's body function performance.

3. The system of claim 1, wherein the computing device is selected from the group consisting of a game console, a laptop, a tablet, a smartphone, and a desktop computer.

4. The system of claim 1, wherein the motion detection camera detects movement of at least twelve separate anatomical points on the user.

5. The system of claim 1, wherein the motion detection camera detects movement in the x-axis, y-axis, and z-axis of at least twelve separate anatomical points on the user.

6. The system of claim 1, wherein the motion detection camera captures a second plurality of body movements from the user's movements after at least one treatment session for storage on a third database of body movements on the data storage device, wherein the system compares the third database of body movements to the first and second database of body movements to determine improvement of the user's body motor function disorder symptoms, and further wherein the system outputs a second unique evaluation of the user's body function performance.

7. The system of claim 6, wherein the motion detection camera captures a third plurality of body movements from the user's movements after at least two treatment sessions for storage on a fourth database of body movements on the data storage device, wherein the system compares the fourth database of body movements to the first, second, and third databases of body movements to determine improvement of the user's body motor function disorder symptoms, and further wherein the system outputs a third unique evaluation of the user's body function performance.

8. The system of claim 1, wherein the motion detection camera captures an N plurality of body movements from the user's movements after at least N−1 treatment sessions, where N is an integer greater than 2, for storage on an N+1 database of body movements on the data storage device, wherein the system compares the N+1 database of body movements to the N database of body movements to determine improvement of the user's body motor function disorder symptoms, and further wherein the system outputs an N unique evaluation of the user's body function performance.

10. The system of claim 1, wherein the body motor function disorder is selected from the group consisting of Autism Spectrum Disorders, Parkinson's Disease, Ataxia, Dystonia, Dyspraxia, body swaying motions, tremors, jerks, twitches, spasms, contractions, and gait problems, speech problems, lack of coordination, and loss of balance.

11. A diagnosis and evaluation system for body motor function disorders, the system comprising:
a computing device comprising executable software;
a data storage device;
a first database body movements on the data storage device, wherein the first database of body movements corresponds to body movement symptoms of at least one body motor function disorder;
a motion detection camera, wherein the motion detection camera detects a user, wherein the user is subjected to a plurality of stimuli in an interactive virtual environment to generate a plurality of body movements from the user, further wherein the motion detection camera captures the plurality of body movements from the user for storage onto a second database of body movements on the data storage device;
wherein the system compares the second database of body movements from the user's plurality of body movements to the first database of body movements to determine if the user is diagnosed with a body motor function disorder, and further wherein the system outputs a first unique evaluation of the user's body function performance;
wherein after at least N−1 treatment sessions, where N is an integer greater than 2, the motion detection camera captures an N plurality of body movements from the user's body movements in response to the plurality of stimuli in the interactive virtual environment for storage on an N+1 database of body movements on the data storage device, wherein the system compares the N+1 database of body movements to the N database of body movements to determine improvement of the user's body motor function disorder symptoms, and further wherein the system outputs an N unique evaluation of the user's body function performance.

12. The system of claim 11, wherein the system generates a unique treatment plan based on the user's body function performance.

13. The system of claim 11, wherein the computing device is selected from the group consisting of a game console, a laptop, a tablet, a smartphone, and a desktop computer.

14. The system of claim 11, wherein the motion detection camera detects movement of at least twelve separate anatomical points on the user.

15. The system of claim 11, wherein the motion detection camera detects movement in the x-axis, y-axis, and z-axis of at least twelve separate anatomical points on the user.

16. The system of claim 11, wherein the body motor function disorder is selected from the group consisting of Autism Spectrum Disorder, Parkinson's Disease, Ataxia, Dystonia, Dyspraxia, body swaying motions, tremors, jerks, twitches, spasms, contractions, and gait problems, speech problems, lack of coordination, and loss of balance.
